(19) United States
(12) Patent Application Publication
(10) Pub. No.: US 2022/0031634 A1
(43) Pub. Date: Feb. 3, 2022

(54) RADIOPROTECTIVE NANODRUG FOR SMALL INTESTINE AND PREPARATION METHOD THEREOF

(71) Applicant: SOOCHOW UNIVERSITY, Suzhou (CN)

(72) Inventors: Daoben HUA, Suzhou (CN); Yushuo ZHANG, Suzhou (CN); Lu WANG, Suzhou (CN); Sen YANG, Suzhou (CN)

(21) Appl. No.: 17/047,547

(22) PCT Filed: Oct. 11, 2019
(86) PCT No.: PCT/CN2019/110565

§ 371 Date: Oct. 14, 2020

(30) Foreign Application Priority Data
Jun. 24, 2019 (CN) 201910550078.3

(51) Int. Cl. A61K 9/51; A61K 9/00; A61K 31/454; A61P 1/00

(52) U.S. Cl. CPC A61K 9/5161; A61P 1/00; A61K 31/454; A61K 9/0053

(57) ABSTRACT

The present invention relates to a method for constructing a nanodrug with adhesion in small intestine, including activating a basic amino acid with a small molecular catalyst, adding a polysaccharide solution and reacting to obtain an amphiphilic polymer; then adding a drug solution and mixing uniformly, to obtain drug-loaded nanoparticles including a hydrophilic portion that is the basic amino acid and a hydrophobic portion that is the polysaccharide and drug, wherein the drug has radioprotective effect or can inhibit ionizing radiation-induced cell death; and adding the drug-loaded nanoparticles to a dopamine solution to obtain a nanodrug including the basic amino acid and polydopamine on the surface after the reaction. The present invention provides an oral nanodrug with adhesion in intestinal tract, and the nanodrug has good biocompatibility, adhesion in small intestine and mucus barrier penetration ability, and can withstand the acid and alkali environment in the gastrointestinal tract.

### FIELD OF THE INVENTION

The present invention relates to a nanodrug for small intestine, and more particularly to a radioprotective nanodrug for small intestine and preparation method thereof.

### DESCRIPTION OF THE RELATED ART

As the rapid development and wide application of the nuclear industry and nuclear technology, the nuclear safety has exhibited its significance in the past several years. Rapid and accurate radioprotection has become an important topic in the research of nuclear safety. Short-term exposure of the body to ionizing radiation greater than 10 Gy will lead to severe gastrointestinal syndrome such as diarrhea, hematochezia, intestinal inflammation even death within a few weeks. Although the currently available radioprotective drugs can give a certain radiation protection property, their low targeting and severe side effects often lead to ineffective treatment of intestinal radiation injury. At present, the radiation protection methods for small intestine mainly include injectable and oral formulations. The injectable radioprotective drugs for small intestine are mainly antioxidants (DOI:10.1016/j.freeradbiomed.2018.10.) and thiol compounds (DOI: 10.1634/theoncologist.12-6-738) for scavenging free radicals generated by ionizing radiation; small molecule drugs (DOI: 10.1053/gast.2002.34209; DOI: 10.1093/jrr/rrs001) and protein-based pharmaceuticals (DOI: 10.1126/science.1154986) for inhibiting radiation-induced apoptosis of intestinal epithelial cells; and cytokine therapy agents (DOI: 10.1084/jem.173.5.1177; DOI: 10.1097/00002820-200308000-00012) and bone marrow cell-derived extracellular vesicles (DOI: 10.1038/ncomms13096) for accelerating the regeneration and reconstruction of the intestinal stem cells. The above drugs are distributed throughout the body through invasive operations (such as intravenous injection, and intraperitoneal injection, etc.) to achieve the radiation protection effect for small intestine. Oral formulations such as fecal microbiota transplantation (DOI: 10.15252/emmm.201606932), and amifostine microcapsules (DOI: 10.1016/j.ijpharm.2013.06.019) and Ex-RAD® (DOI: 10.1269/jrr.11191) have to reside in the intestinal lumen, or cross the intestinal epithelium to reach systemic circulation to exert a radiation protection effect on the small intestine. However, the gastrointestinal peristalsis, the rapid clearance of intestinal fluid, and harsh environment during gastrointestinal tract passage make it difficult for oral formulations to reside in the gastrointestinal tract chronically and effectively, limiting the radiation protection effect of oral formulations. At present, there is no nanodrug that can adhere to the small intestine tissue in the liquid environment of the gastrointestinal tract and slowly controlled release the drug to achieve the radiation protection effect.

Injectable drugs may cause serious toxic side effects (for example, protein-based pharmaceuticals can induce immune responses in the body), and may be difficult to ensure that the drugs can reach the small intestinal tissue, which is highly sensitive to radiation (where studies have found that drugs often tend to concentrate in the liver and spleen). Oral formulations may be broken down by the extreme pH environment and digestive enzymes in the gastrointestinal tract. Most importantly, most of the drugs are unable to withstand the rapid clearance of intestinal fluid. Therefore, oral drugs are often difficult to reside in the vulnerable small intestine tissue, and thus cannot exhibit effective radiation protection.

### SUMMARY OF THE INVENTION

To solve these problems, the invention provides a radioprotective nanodrug for small intestine as well as an efficient preparation method thereof. Particularly, a nanodrug with adhesion to intestinal conditions is provided, which can withstand the harsh environment in the gastrointestinal tract, good biocompatibility and the ability to approach the small-intestine mucus barrier, and effective enrichment in the intestinal crypt stem cells.

A first object of the present invention is to provide a method for preparing a nanodrug, which comprises the following steps:

(1) A hydrophilic basic amino acid was activated with a small-molecule catalyst in an acidic buffer solution. Then a polysaccharide solution was added and mixed uniformly. The mixture was reacted at pH 4.5-5.5 and 20-30° C. to obtain an amphiphilic macromolecular polymer. After a drug solution was added and mixed uniformly, drug-loaded nanoparticles were obtained to comprise a hydrophilic portion to give the basic amino acid and a hydrophobic portion as the polysaccharide and drug with radiation protection effect or inhibiting ionizing radiation-induced cell death effect (such as apoptosis, and pyroptosis, etc.).

(2) adding the drug-loaded nanoparticles obtained in Step (1) to a dopamine solution, and reacting at pH 8.0-10.0 and 25-50° C., to obtain a nanodrug comprising the basic amino acid and polydopamine on the surface after the reaction is complete.

Preferably, in Step (1), the acidic buffer solution is morpholinoethanesulfonic acid, glacial acetic acid or hydrochloric acid.

Preferably, in Step (1), the activation time is 2-6 h.

Preferably, in Step (1), the most preferable molar ratio of the small-molecular catalyst (N-hydroxysuccinimide and 1-(3-dimethylaminopropyl)-3-ethylcarbodiimide hydrochloride) is given as 1:1.

Preferably, in Step (1), the molar ratio of the basic amino acid, N-hydroxysuccinimide and 1-(3-dimethylaminopropyl)-3-ethylcarbodiimide hydrochloride is 1:4:4.

Preferably, in Step (1), the basic amino acid is arginine, lysine, histidine, et al.

Preferably, in Step (1), the polysaccharide is a hydrophobic polysaccharide, which is preferably chitosan, dextran, alginic acid, cellulose, etc. The molar ratio of the carboxyl group of the basic amino acid and the amino group of the polysaccharide is 1:1. The molecular weight of the polysaccharide is preferably 20-200 kD, where the degree of deacetylation of chitosan is 75%-95%.

Preferably, in Step (1), the solvent of the polysaccharide solution is morpholinoethanesulfonic acid.

Preferably, in Step (1), during the preparation of the amphiphilic polymer, the reaction solution is stirred continuously for 24-48 h with the pH maintained at 4.5-5.5 by buffer solution. After the reaction is completed, an alkaline solution is added to terminate the reaction.

In the prepared amphiphilic polymer in Step (1), the basic amino acid and the polysaccharide are covalently bonded. Since the molecular weight of the polysaccharide is much larger than that of the basic amino acid, the polysaccharide forms the internal structure of the nanoparticles, and the basic amino acid is evenly distributed outside of the nanoparticles.

Preferably, in Step (1), the drug is a hydrophobic drug, and preferably thalidomide, cysteamine thiosulfate, amifostine, genistein, resveratrol, 3,3-diindolylmethane, Entolimod, and Ex-RAD, etc. The concentration of the drug solution is 1.0 mg/mL, and the weight ratio of the drug to the polysaccharide encapsulated by the amino acid is 1:100.

Preferably, in Step (1), the solvent of the drug solution is a mixed solvent of water and an organic solvent at a volume ratio of 1:1, and the organic solvent is preferably acetonitrile.

Preferably, in Step (1), during the preparation of the drug-loaded nanoparticles, the reaction is performed with continuous stirring for 24 h under a protective atmosphere. After the reaction is complete, the solvent is removed, and the remainder is centrifuged, and lyophilized. More preferably, in Step (2), the concentration of the dopamine solution is 2.0 mg/mL; and the weight ratio of the nanoparticles to dopamine is 1:4.

Preferably, in Step (2), the reaction is performed with stirring for 3-12 h.

A second object of the present invention is to provide a nanodrug prepared by the above preparation method. The nanodrug includes nanoparticles and polydopamine modified on the surface of the nanoparticles. The nanoparticles comprise a hydrophobic polysaccharide, a hydrophilic basic amino acid and a hydrophobic drug. The polysaccharide and the basic amino acid are covalently bonded, and the drug is located inside the nanoparticles and is positively charged in the gastric acid environment. The particle size of the nanodrug is 100-500 nm.

In gastric acid, the surface of the nanodrug is positively charged (due to protonation of the polydopamine and basic amino acid on the surface), and the small molecule drug carried inside the nanoparticles is also positively charged, which cannot be released due to the charge repulsion. After reaching the small intestine, due to the almost neutral surface of the nanodrug (since the negative charge resulting from hydroxyl deprotonation of dopamine neutralizes the positive charge of the basic amino acid), the charge repulsion is relieved, so that the drug inside the nanoparticles is slowly released, ensuring that the drug is largely released in the small intestine that is highly sensitive to radiation.

The drug prepared in the present invention has the characteristics of being more suitable for penetrating the network structure of the small-intestine mucus barrier, and can quickly reach the intestinal crypt site under the mucus barrier, which is most vulnerable to radiation damage in the small intestine. At present, there is no radioprotective drug for small intestine that can be directly delivered to the crypt by oral administration.

A third object of the present invention is to claim the use of the above-mentioned nanodrug of the present invention in the radiation protection of small intestine.

Preferably, the preparation is an oral drug.

Preferably, the radiation is X-ray radiation, or γ-ray radiation (⁶⁰Co source or ¹³⁷Cs source).

Preferably, the radiate position is the abdomen, and the radiation dose is 2-15 Gy.

Preferably, the protection preparation has good adhesivity to small intestinal crypts and the best radiation protection effect.

The preparation principle of the nanodrug of the present invention is as follows:

After the activated basic amino acid is mixed with the polysaccharide, amidation occurs in the buffer solution, so that the carboxyl group of the amino acid is linked to the amino group on the polysaccharide to form an amphiphilic polymer. Because the molecular weight of the polysaccharide is much higher than that of the amino acid, the polysaccharide forms a random coil-like structure, and the amino acid is distributed outside the random coil-like structure. Then the solution of the drug in a mixed solvent of an organic solvent and water is slowly added dropwise. Since the drug and the polysaccharide are both hydrophobic, the drug will be encapsulated inside the amphiphilic polymer according to the principle of “like dissolves like”, and forms nanoparticles which have a hydrophobic interior and a hydrophilic surface. In an alkaline aqueous solution, dopamine undergoes oxidative self-polymerization, and the formed polydopamine tends to coat the surface of the nanoparticles in the aqueous environment containing the drug-loaded nanoparticles to form a polydopamine coating structure, thereby forming the nanodrug.

By means of oral administration, the present invention can achieve the drug delivery to the small intestine. While the local effective concentration of the drug is increased by means of the adhesion of polydopamine, the systemic side effects of the drug is reduced. After oral administration, the polydopamine modified on the surface of the nanodrug allows for adhesion to the intestine. In the small intestine, the polysaccharide in the nanodrug swells after absorbing water, and slowly releases the drug.

By means of the above technical solutions, the present invention has the following advantages.

(1) The present invention provides a nanodrug for small intestine. Similar concept can be used to optimize other route of administration to the small intestine, which is different from conventional intravenous and oral administration. The nanodrug can withstand the acid and alkali environment of the gastrointestinal tract, and has good biocompatibility. The suitable particle size and surface charge of the nanodrug make it have the ability to penetrate the small-intestine mucus barrier, and the nanodrug has adhesion ability in the intestinal fluid environment, and can significantly prolong the time of drug for the small intestine and improve the utilization efficiency.

(2) Oral radioprotective nanodrug is used for radiation protection of small intestine, in which the radioprotective drug can be efficiently and stably delivered to the small intestinal crypt region to ensure that the drug directly acts on the stem cells in small intestinal crypt with high radiation sensitivity.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 is an SEM image of the prepared radioprotective nanodrug;

FIG. 2 shows the result of the hydrodynamic particle size of the radioprotective nanodrug;

FIG. 3 shows the standard ultraviolet absorption curve of the loaded drug thalidomide and the test result of the drug loading rate of the nanodrug;

FIG. 4 is a schematic diagram of the radioprotective nanodrug;

FIG. 5 shows the test result of in-vitro radiation protection effect of the radioprotective nanodrug;

FIG. 6 shows the test result of adhesion in intestinal tract of the radioprotective nanodrug;

FIG. 7 is the mode of action of the radioprotective nanodrug delivered by oral administration; and

FIG. 8 shows the effect of the radioprotective nanodrug in attenuation of radiation-induced intestinal damage.

### REFERENCE NUMERALS

1—chitosan; 2—arginine; 3—thalidomide; 4—polydopamine; 5—radioprotective nanodrug; 6—small intestinal mucus barrier; 7—intestinal villi; 8—intestinal crypt.

### DETAILED DESCRIPTION OF THE PREFERRED EMBODIMENTS

The specific embodiments of the present invention will be described in further detail with reference to the accompanying drawings and examples. The following examples are intended to illustrate the present invention, instead of limiting the scope of the present invention.

### Example 1: Synthesis of Nanodrug

Arginine (0.867 g, 4.977 mmol) was dissolved in morpholinoethanesulfonic acid (40 mL, 25 mM, pH 5.0). Then, N-hydroxy succinimide (2.291 g, 19.908 mmol), and 1-(3-dimethylaminopropyl)-3-ethylcarbodiimide hydrochloride (3.816 g, 19.908 mmol) were added in sequence for activation for 2 h. Subsequently, a solution of chitosan (1.0 g, 4.977 mmol) dissolved in morpholinoethanesulfonic acid was added to the mixture, and reacted for 24 h with continuous stirring at room temperature. Sodium hydroxide (0.1 M) was then added to terminate the reaction. Subsequently, thalidomide (1.0 mg/mL, 10 mL) dissolved in a mixed solution of water and acetonitrile (v/v=1/1) was slowly added dropwise to the polymer solution (10 mg/mL, 100 mL). The resulting solution was continuously stirred and nitrogen was introduced overnight. Acetonitrile was removed, and the supernatant was collected and lyophilized after centrifugation. The lyophilized sample (20.0 mg) was transferred to a dopamine solution (2 mg/mL, 40 mL, pH 8.5), stirred at room temperature for 3 h, washed with deionized water, and centrifuged. The supernatant was collected to obtain a nanodrug.

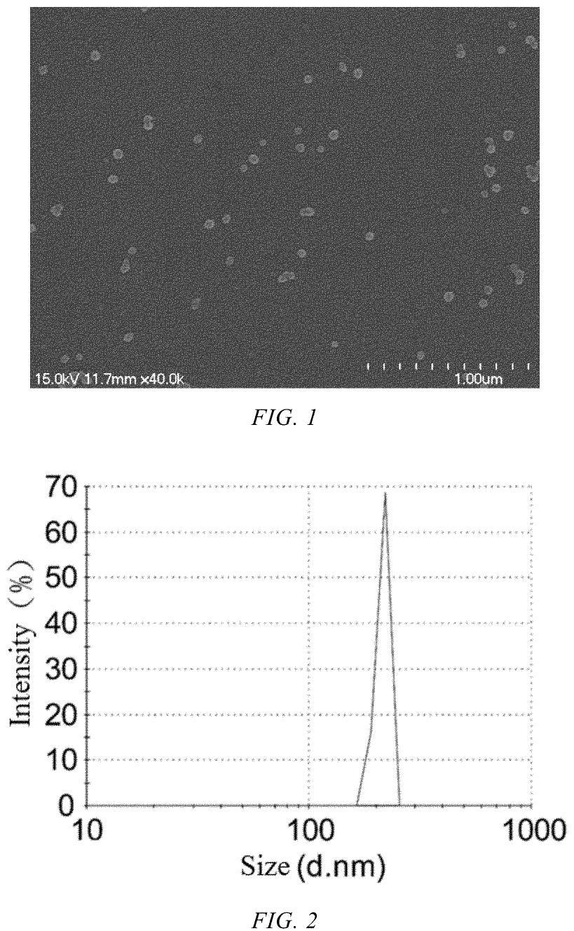

FIG. 1 is an SEM image of the prepared radioprotective nanodrug. The result shows that the nanodrug has a small particle size and good dispersion. The nano-drug has a nearly spherical structure with a relatively smooth surface. It indicates that polydopamine is evenly coated on the surface of the nanoparticles by forming a coating.

FIG. 2 shows the test result of the hydrodynamic diameter of the radioprotective nanodrug. The result shows that the nanodrug has a small hydrodynamic diameter of about 214 nm and a PDI of 0.584. The particle size is suitable for penetrating the small intestinal mucus barrier, and facilitates the nanodrug to exert a radiation protection effect in the small intestinal crypt site.

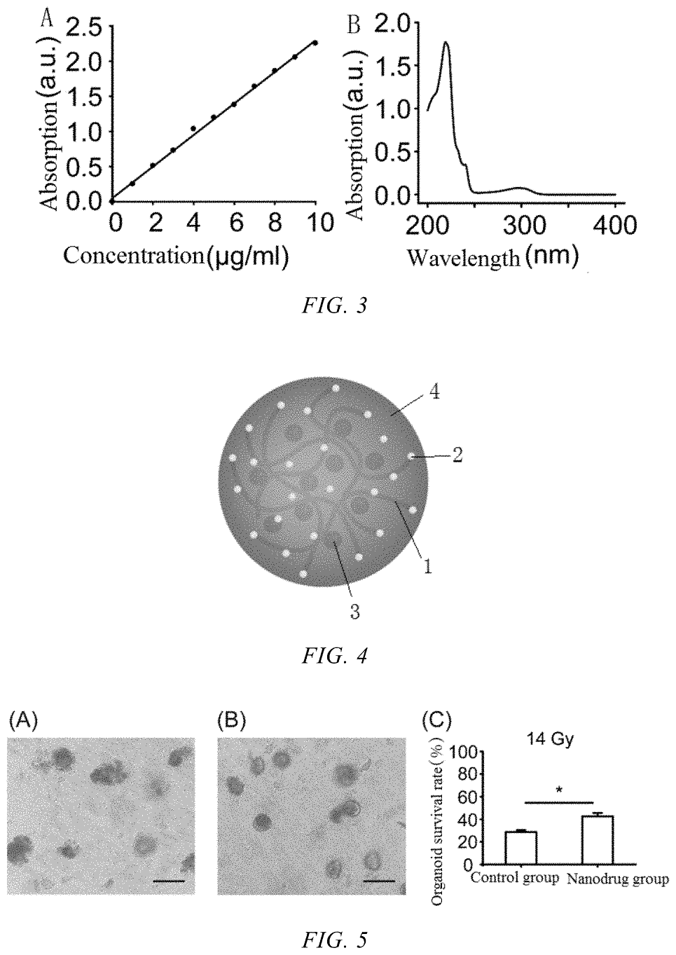

FIG. 3 shows the standard ultraviolet absorption curve of the loaded thalidomide and the test result of the drug loading rate of the radioprotective nanodrug. By substituting the UV absorbency (FIG. 3B) of the ultrasonicated nanodrug solution into the calculation formulation (FIG. 3A, y=0.2245x+0.051, R²=0.9975) from the standard curve, the drug loading rate of the nanodrug is calculated to be about 22.98%.

FIG. 4 is a schematic diagram showing the structure of the prepared radioprotective nanodrug, including chitosan 1, arginine 2, thalidomide 3, and polydopamine 4. Chitosan 1 forms a network structure having a surface to which arginine 2 is linked, thalidomide 3 is enveloped in the network structure, and polydopamine 4 is located on the surface of the nanodrug.

### Example 2. In-Vitro Radiation Protection Effect Test

An appropriate amount of nanodrug (11.237 μg/mL) prepared in Example 1 were dispersed in a medium for culturing small intestinal crypt organoid, and C57BL/6J mouse small intestinal crypt organoids were cultured ex vivo, and irradiated with X ray at a dose of 14 Gy after 12 h. After radiation damage, the disintegrated small intestinal crypts (as shown in FIG. 5A) and the intact crypts with sharp edge (as shown in FIG. 5B) were calculated. As shown in FIG. 5C, the survival rate of the crypts treated with the nanodrug after irradiation is about 42.67%, which is significantly higher than that of the control group (*p<0.05). The result shows that the nanodrug has good radiation protection effect.

### Example 3. Test of Adhesion in Intestinal Tract

The radioprotective nanodrug prepared in Example 1 was labeled with Cy5.5 fluorescent dye, and then resuspended in phosphate buffer solution. C57BL/6J mice were fasted for 12 h, and the dye-labeled nanodrug solution (4 mg/mL, 0.5 mL) was administered to mice in each group by intragastric administration. The mice were euthanized 6 h and 24 h after administration, and the small intestine tissues were taken for in-vitro fluorescence imaging. The instrument used was Kodak FX Pro in-vivo fluorescence imaging system. The wavelength of the excitation light was 630 nm and the wavelength of the emission light was 700 nm.

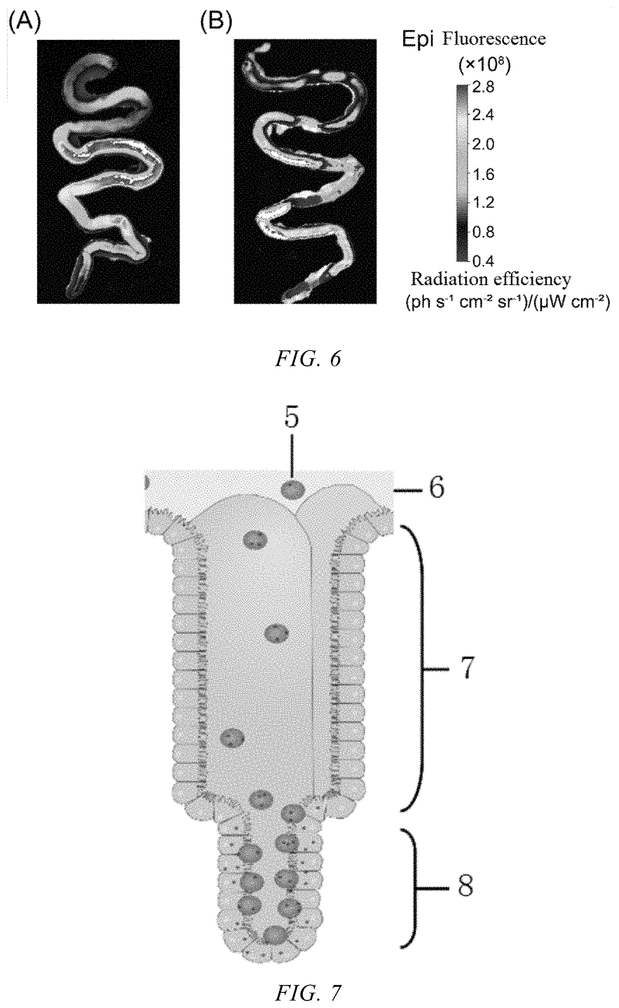

As shown in FIG. 6, 6 h after administration (FIG. 6A), the small intestine of mice shows a strong fluorescent signal, indicating that the drug has mostly been accumulated in the small intestine. 24 h after administration (FIG. 6B), the fluorescent signal in the small intestine tissue of the mice remains at a strong level, indicating that the nanodrug has good adhesion in small intestine.

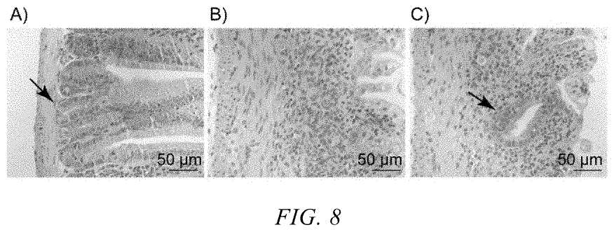

FIG. 7 is a schematic diagram showing the mode of action of the radioprotective nanodrug delivered by oral administration. The radioprotective nanodrug 5 can withstand the acid and alkali environment in the gastrointestinal tract and thus can be avoided from decomposition and absorption into the blood under the action of gastric acid. Due to its adhesion ability in the intestinal fluid environment, the nanodrug can penetrate the small intestinal mucus barrier 6 to reach the intestinal villi 7, and further reach the small intestinal crypt 8.

### Example 4. Test of Ability to Relieve Radiation-Induced Intestinal Damage

The radioprotective nanodrug (22.98 wt. %, containing 100 mg/kg thalidomide in 500 μL phosphate buffer) was administered to C57BL/6J mice (male, 8 weeks old) by intragastric administration 12 h before irradiation, and the simple irradiation group was given the same dose of phosphate buffer. The X-RAD 320i X-ray machine was used to irradiate the abdomen of the mice at a dose of 14 Gy and a dose rate of 1 Gy/min. 5 days after irradiation, the small intestine tissues of the mice were taken to make paraffin sections which were stained with hematoxylin-eosin. The main evaluation criterion of radiation-induced intestinal damage includes the number of pathologically detected crypts in the intestinal sample. The regeneration and repair of the intestinal tract after irradiation mainly rely on the stem cells in the crypt site, so the survival and intactness of the small intestinal crypts after irradiation can reflect the severity of radiation damage in the small intestine.

As shown in FIG. 8, the normal small intestinal crypt structure is shown in FIG. 8A, and the crypt structures almost disappear in the simple irradiation group 5 days after irradiation (as shown in FIG. 8B), indicating that ionizing radiation causes severe intestinal damage and the highly reduced crypts cannot exert the regeneration and repair function, which leads to the death of the mice. In the group treated with the radioprotective nanodrug (FIG. 8C), some small intestinal crypts still retain the original contour and regenerate 5 days after the irradiation (as shown in FIG. 8C, the arrow indicates the viable crypts), indicating that the small intestine tissue still has the repair and regeneration ability after irradiation, and thus the radioprotective nanodrug can greatly relieve radiation-induced intestinal injury.

While preferred embodiments of the present invention have been described above, the present invention is not limited thereto. It should be appreciated that some improvements and variations can be made by those skilled in the art without departing from the technical principles of the present invention, which are also contemplated to be within the scope of the present invention.

1. A method for preparing a nanodrug for small intestine, comprising the following steps:
(1) activating a hydrophilic basic amino acid with a small-molecule catalyst in an acidic buffer solution, then adding a polysaccharide solution, mixing uniformly and reacting at pH 4.5-5.5 and 20-30° C. to obtain an amphiphilic high-molecular polymer; then adding a drug solution and mixing uniformly, to obtain drug-loaded nanoparticles comprising a hydrophilic portion that is the basic amino acid and a hydrophobic portion that is the polysaccharide and drug, wherein the drug has radiation protection effect or has the effect of inhibiting ionizing radiation-induced cell death, and the drug is positively charged in agastricacid environment; and
(2) adding the drug-loaded nanoparticles obtained in Step (1) to a dopamine solution, and reacting at pH 8.0-10.0 and 25-50° C., to obtain a nanodrug having a surface modified with polydopamine after the reaction is complete.

2. The preparation method according to claim 1, wherein in Step (1), the small-molecular catalyst is N-hydroxysuccinimide and 1-(3-dimethylaminopropyl)-3-ethylcarbodiimide hydrochloride.

3. The preparation method according to claim 2, wherein in Step (1), the molar ratio of the basic amino acid, N-hydroxysuccinimide and 1-(3-dimethylaminopropyl)-3-ethylcarbodiimide hydrochloride is 1:4:4.

4. The preparation method according to claim 1, wherein in Step (1), the basic amino acid is selected from arginine, lysine, histidine and any combination thereof.

5. The preparation method according to claim 1, wherein in Step (1), the polysaccharide is selected from the group consisting of chitosan, dextran, alginic acid, cellulose and any combination thereof; and the molar ratio of the carboxyl group of the basic amino acid to the amino group of the polysaccharide is 1:1.

6. The preparation method according to claim 1, wherein in Step (1), the drug is selected from the group consisting of thalidomide, cysteamine thiosulfate, amifostine, genistein, resveratrol, 3,3-diindolylmethane, Entolimod, and Ex-RAD and any combination thereof; the concentration of the drug solution is 1.0 mg/mL; and the weight ratio of the drug to the polysaccharide encapsulated by the amino acid is 1:100.

7. The preparation method according to claim 1, wherein in Step (2), the concentration of the dopamine solution is 2.0 mg/mL; and the weight ratio of the nanoparticles to dopamine is 1:4.

8. A nanodrug for small intestine prepared by the preparation method according to claim 1, comprising nanoparticles and polydopamine modified on the surface of the nanoparticles, wherein the nanoparticles comprise a hydrophobic polysaccharide, a hydrophilic basic amino acid and a hydrophobic drug, wherein the polysaccharide and the basic amino acid are covalently bonded, the drug is located inside the nanoparticles and is positively charged in agastricacid environment, and the nanodrug has a particle size of 100-500 nm.

9. Use of the nanodrug according to claim 8 in the production of a radioprotective agent for small intestine.

10. The use according to claim 9, wherein the radioprotective agent is an oral drug.
